(19) United States
(12) Patent Application Publication
(10) Pub. No.: US 2016/0058292 A1
(43) Pub. Date: Mar. 3, 2016

(54) Photoacoustic Imager

(71) Applicant: XTrillion, Inc., Tokyo (JP)

(72) Inventor: Hitoshi NAKATSUKA, Tokyo (JP)

(21) Appl. No.: 14/790,450

(22) Filed: Jul. 2, 2015

(30) Foreign Application Priority Data
Aug. 27, 2014 (JP) 2014-172532

(51) Int. Cl. A61B 5/00

(52) U.S. Cl. CPC A61B 5/0095; A61B 2560/0266

(57) ABSTRACT

This photoacoustic imager includes a light-emitting element, an acoustic wave detection portion and a light source driving portion, and the light source driving portion is configured to substantially null the value of current flowing in the light-emitting element by stopping supplying power to the light-emitting element on the basis of that the value of the current flowing in the light-emitting element has reached a prescribed current value.

### BACKGROUND OF THE INVENTION

1. Field of the Invention

The present invention relates to a photoacoustic imager, and more particularly, it relates to a photoacoustic imager including a light-emitting element emitting light to be applied to a specimen.

2. Description of the Background Art

A photoacoustic imager including a light-emitting element emitting light to be applied to a specimen is known in general, as disclosed in Japanese Patent Laying-Open No. 2010-42158, for example.

The aforementioned Japanese Patent Laying-Open No. 2010-42158 discloses a photoultrasonic tomographic imager including a semiconductor laser pulse light source applying pulsed light to a tested portion through a fiber amplifier and an ultrasonic wave detection means. This photoultrasonic tomographic imager is configured to apply the pulsed light from the semiconductor laser pulse light source to the tested portion through the fiber amplifier as measurement light and to detect an ultrasonic wave generated by the tested portion due to the applied measurement light with the ultrasonic wave detection means.

In the photoultrasonic tomographic imager according to the aforementioned Japanese Patent Laying-Open No. 2010-42158, however, there may conceivably be a period when the magnitude of current flowing in the semiconductor laser pulse light source remains unchanged when the semiconductor laser pulse light source emits the pulsed light. In a period when the magnitude of light absorbed by a detection object in the tested portion (a specimen) remains unchanged, the detection object generates no ultrasonic wave (acoustic wave), and hence power consumption for emitting light conceivably disadvantageously increases in the photoultrasonic tomographic imager according to the aforementioned Japanese Patent Laying-Open No. 2010-42158, due to emission of light not contributing to generation of the acoustic wave.

### SUMMARY OF THE INVENTION

The present invention has been proposed in order to solve the aforementioned problem, and an object of the present invention is to provide a photoacoustic imager capable of preventing increase in power consumption for emitting light by preventing emission of light not contributing to generation of an acoustic wave.

In order to attain the aforementioned object, a photoacoustic imager according to an aspect of the present invention includes a light-emitting element emitting light to be applied to a specimen, an acoustic wave detection portion detecting an acoustic wave generated by a detection object in the specimen absorbing the light applied from the light-emitting element to the specimen and a light source driving portion supplying power for making the light-emitting element emit the light to the light-emitting element, while the light source driving portion is configured to substantially null the value of current flowing in the light-emitting element by stopping supplying the power to the light-emitting element on the basis of that the value of the current flowing in the light-emitting element has reached a prescribed current value.

In the photoacoustic imager according to the aspect of the present invention, as hereinabove described, the light source driving portion is configured to stop supplying the power to the light-emitting element on the basis of that the value of the current flowing in the light-emitting element has reached the prescribed current value. Thus, the light source driving portion can stop supplying the power to the light-emitting element before the value of the current flowing in the light-emitting element reaches a substantially unchanged current value, whereby a period when the magnitude of the current flowing in the light-emitting element remains unchanged can be shortened or eliminated. Further, emission of light not contributing to generation of the acoustic wave can be prevented by shortening or eliminating the period when the magnitude of the current flowing in the light-emitting element remains unchanged. Consequently, the photoacoustic imager can prevent increase in power consumption for emitting the light by preventing emission of the light not contributing to generation of the acoustic wave. Further, the photoacoustic imager can also prevent heat generation resulting from power consumption by preventing increase in power consumption.

In the photoacoustic imager according to the aforementioned aspect, the light source driving portion is preferably configured to start supplying the power to the light-emitting element in a state where the value of the current flowing in the light-emitting element is substantially zero and to stop supplying the power to the light-emitting element on the basis of that the value of the current flowing in the light-emitting element has reached the prescribed current value so that the waveform of the current flowing in the light-emitting element becomes triangular. According to this structure, the period when the magnitude of the current flowing in the light-emitting element remains unchanged can be further shortened as compared with a case of making the waveform of the current flowing in the light-emitting element rectangular, by making the waveform of the current flowing in the light-emitting element triangular. Consequently, the photoacoustic imager can further prevent increase in power consumption for emitting light by preventing emission of light not contributing to generation of the acoustic wave.

In the photoacoustic imager according to the aforementioned aspect, the light source driving portion is preferably configured to change the value of the current flowing in the light-emitting element substantially in all periods for feeding the current to the light-emitting element. According to this structure, the period when the magnitude of the current flowing in the light-emitting element remains unchanged is substantially eliminated from the periods for feeding the current to the light-emitting element, whereby the photoacoustic imager can further prevent emission of light not contributing to generation of the acoustic wave.

In this case, the periods for feeding the current to the light-emitting element preferably consist of a first period when the value of the current flowing in the light-emitting element increases from a substantially zero state and a second period when the value of the current flowing in the light-emitting element decreases from the prescribed current value. According to this structure, the photoacoustic imager can be easily configured to change the value of the current flowing in the light-emitting element substantially in all periods for feeding the current to the light-emitting element.

In the aforementioned photoacoustic imager having the first and second periods, the first period preferably corresponds to a rise time of the light-emitting element, and the second period preferably corresponds to a fall time of the light-emitting element. According to this structure, the photoacoustic imager can easily set the first period when the value of the current flowing in the light-emitting element increases from the substantially zero state and the second period when the value of the current flowing in the light-emitting element decreases from the prescribed current value by making the first and second periods correspond to the rise time and the fall time of the light-emitting element respectively.

In the photoacoustic imager according to the aforementioned aspect, the light-emitting element is preferably configured to emit pulsed light having a triangular waveform by being supplied with the power from the light source driving portion. According to this structure, the photoacoustic imager can prevent emission of light not contributing to generation of the acoustic wave since the pulsed light having a triangular waveform has no period when the intensity of the light remains unchanged. Consequently, the photoacoustic imager can prevent increase in power consumption for emitting light.

The photoacoustic imager according to the aforementioned aspect preferably further includes a current detection portion detecting the value of the current flowing in the light-emitting element by detecting the magnitude of a voltage drop in voltage applied to the light-emitting element due to the supply of the power from the light source driving portion. Generally in a case of detecting the value of current flowing in a light-emitting element, a peak holding circuit including a detection resistor, a charge capacitor and a switch must be provided, for example. On the other hand, the value of the current flowing in the light-emitting element is correlated with the magnitude of a voltage drop in voltage applied to the light-emitting element. When the current detection portion is configured to acquire the value of the current flowing in the light-emitting element by detecting the magnitude of the voltage drop in the voltage applied to the light-emitting element as described above, therefore, the same can acquire the value of the current flowing in the light-emitting element with no requirement for a peak holding circuit or the like. The structure of detecting the magnitude of the voltage drop in the voltage applied to the light-emitting element is uncomplicated as compared with a peak holding circuit, whereby the photoacoustic imager can be prevented from complication in structure.

In this case, the current detection portion is preferably configured to acquire the value of the current flowing in the light-emitting element on the basis of the magnitude of the voltage drop in the voltage and a current characteristic of the light-emitting element corresponding to the magnitude of the voltage drop in the voltage. According to this structure, the current detection portion can easily acquire (calculate) the value of the current flowing in the light-emitting element with the current characteristic of the light-emitting element corresponding to the magnitude of the voltage drop in the voltage by detecting the magnitude of the voltage drop in the voltage.

In the aforementioned photoacoustic imager acquiring the value of the current flowing in the light-emitting element on the basis of the current characteristic of the light-emitting element corresponding to the magnitude of the voltage drop in the voltage, the current characteristic of the light-emitting element corresponding to the magnitude of the voltage drop in the voltage is preferably a characteristic associating a forward voltage value of the light-emitting element and the value of the current flowing in the light-emitting element with each other. It is generally known that a forward voltage value of a light-emitting element and the value of current flowing in the light-emitting element are correlated with each other. Further, the magnitude of a voltage drop per light-emitting element and the forward voltage value of the light-emitting element substantially coincide with each other. Noting these points, the photoacoustic imager according to the present invention can easily acquire the value of the current flowing in the light-emitting element by employing the magnitude of the voltage drop in the voltage and the characteristic associating the forward voltage value of the light-emitting element and the value of the current flowing in light-emitting element with each other.

In the photoacoustic imager according to the aforementioned aspect, the light source driving portion is preferably configured to supply a driving pulse based on a table including a voltage value corresponding to the prescribed current value and a pulse width corresponding to the prescribed current value to the light-emitting element. According to this structure, the light source driving portion can be configured to stop supplying the power to the light-emitting element at the time when the value of the current flowing in the light-emitting element reaches the prescribed current value without acquiring the value of the current flowing in the light-emitting element, by supplying the driving pulse based on the table to the light-emitting element. Consequently, the photoacoustic imager may be provided with no current detection portion for acquiring the value of the current flowing in the light-emitting element, whereby the same can be further prevented from complication in structure.

In this case, the light-emitting element preferably includes a first light-emitting element emitting light having a first wavelength and a second light-emitting element emitting light having a second wavelength different from the first wavelength, and the light source driving portion is preferably configured to supply the driving pulse to the first light-emitting element on the basis of the table corresponding to the first light-emitting element and to supply the driving pulse to the second light-emitting element on the basis of the table corresponding the second light-emitting element. According to this structure, the photoacoustic imager may be provided with no current detection portions for acquiring the values of current flowing in the light-emitting elements respectively also in the case of providing the first and second light-emitting elements, whereby the light source driving portion can supply driving pulses corresponding to the respective light-emitting elements while preventing the photoacoustic imager from complication in structure.

In the photoacoustic imager according to the aforementioned aspect, a plurality of the light-emitting elements are preferably provided, and preferably serially connected with each other thereby forming a plurality of light-emitting element groups, and the light source driving portion preferably includes a plurality of driving switch portions provided on the respective ones of the plurality of light-emitting element groups. According to this structure, the photoacoustic imager can properly switch states of applying and not applying light from the light-emitting element groups with the driving switch portions provided thereon respectively also when the characteristics (those of forward voltage values, for example) are dispersed among the plurality of light-emitting element groups.

In the photoacoustic imager according to the aforementioned aspect, a plurality of the light-emitting elements are preferably provided, and preferably serially connected with each other thereby forming a plurality of light-emitting element groups, the plurality of light-emitting element groups are preferably parallelly connected to the light source driving portion respectively, the photoacoustic imager preferably further includes a current detection portion acquiring the values of current flowing in the respective ones of the plurality of light-emitting element groups, and the light source driving portion is preferably configured to substantially null the values of the current flowing in the light-emitting elements by stopping supplying the power to the light-emitting elements at a time when the value of current flowing latest in the light-emitting groups reaches the prescribed current value among times when the values of the current flowing in the respective ones of the plurality of light-emitting element groups reach the prescribed current value. According to this structure, the photoacoustic imager can be so configured that all light-emitting element groups reach the prescribed current value by stopping supplying the power on the basis of rise of a light-emitting element group rising latest, also when speeds (response speeds) at which current rises upon supply of voltage to the plurality of light-emitting element groups are dispersed. Thus, quantities of light emitted from the plurality of light-emitting element groups can be ensured, whereby intensity of the acoustic wave can be ensured. Consequently, the photoacoustic imager can more correctly image the acoustic wave when imaging the same.

In the photoacoustic imager according to the aforementioned aspect, the light-emitting element is preferably constituted of a light-emitting diode element. According to this structure, the light-emitting diode element is lower in directivity as compared with a light-emitting element emitting a laser beam, and a light emission range remains relatively unchanged also when misregistration takes place. Thus, the photoacoustic imager requires neither precise alignment (registration) of optical members nor an optical platen or a strong housing for preventing characteristic fluctuation resulting from vibration of an optical system. Consequently, the photoacoustic imager can be prevented from size increase and complication in structure due to the nonrequirement for precise alignment of optical members and an optical platen or a strong housing.

In the photoacoustic imager according to the aforementioned aspect, the light-emitting element is preferably constituted of a semiconductor laser element. According to this structure, the semiconductor laser element can apply a laser beam having relatively high directivity to the specimen as compared with a light-emitting diode element, whereby most part of light from the semiconductor laser element can be reliably applied to the specimen.

In the photoacoustic imager according to the aforementioned aspect, the light-emitting element is preferably constituted of an organic light-emitting element. According to this structure, a probe portion including the organic light-emitting diode element can be easily miniaturized by employing the organic light-emitting diode element easily reducible in thickness.

In the photoacoustic imager according to the aforementioned aspect, the light-emitting element is preferably configured to emit pulsed light having a wavelength in the infrared region. According to this structure, the light having the wavelength in the infrared region can relatively easily penetrate a human body, whereby the photoacoustic imager can deliver the light from the light-emitting element to a deeper portion of the specimen when the specimen is prepared from a human body.

The photoacoustic imager according to the aforementioned aspect preferably further includes a current detection portion detecting the value of current flowing in the light-emitting element, and the current detection portion preferably includes a detection resistor, a capacitor and a detection switch portion. According to this structure, the detection resistor, the capacitor and the detection switch portion can constitute a peak holding circuit, whereby the current detection portion can easily acquire the value (peak) of the current flowing in the light-emitting element by employing the peak holding circuit.

The photoacoustic imager according to the aforementioned aspect preferably further includes a current detection portion detecting the value of current flowing in the light-emitting element, and the current detection portion preferably includes a detection resistor, a capacitor and a diode element. According to this structure, the detection resistor, the capacitor and the diode element can constitute a peak holding circuit. Further, the capacitor and the diode element can maintain a peak value of the current flowing in the light-emitting element, whereby driving of a switch portion may not be controlled dissimilarly to a case of configuring the current detection portion to maintain the peak value of the current flowing in the light-emitting element with the capacitor and a switch portion. Consequently, the photoacoustic imager can be prevented from complication in structure related to control.

In the photoacoustic imager according to the aforementioned aspect, the light source driving portion preferably includes a driving switch portion, and the photoacoustic imager preferably further includes a comparator configured to stop supplying the power from the light source driving portion to the light-emitting element by turning off the driving switch portion when the value of the current flowing in the light-emitting element reaches the prescribed current value. According to this structure, the comparator can substantially null the value of the current flowing in the light-emitting element by automatically stopping supplying the power to the light-emitting element on the basis of that the value of the current flowing in the light-emitting element has reached the prescribed current value.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a block diagram showing the overall structure of a photoacoustic imager according to a first embodiment of the present invention;

FIG. 2 is a block diagram showing the structure of part of the photoacoustic imager according to the first embodiment of the present invention;

FIG. 3 is a diagram for illustrating operations of a current detection portion according to the first embodiment of the present invention;

FIG. 4 is a timing chart for illustrating operations of a conventional photoacoustic imager;

FIG. 5 is a timing chart for illustrating operations of the photoacoustic imager according to the first embodiment of the present invention;

FIG. 6 is a block diagram showing the overall structure of a photoacoustic imager according to a second embodiment of the present invention;

FIG. 7 is a block diagram showing the structure of part of the photoacoustic imager according to the second embodiment of the present invention;

FIG. 8 is a diagram for illustrating characteristics of light-emitting diode elements according to the second embodiment of the present invention;

FIG. 9 is a block diagram showing the overall structure of a photoacoustic imager according to a third embodiment of the present invention;

FIG. 10 is a block diagram showing the structure of part of the photoacoustic imager according to the third embodiment of the present invention;

FIG. 11 is a diagram for illustrating a table according to the third embodiment of the present invention;

FIG. 12 is a block diagram showing the overall structure of a photoacoustic imager according to a fourth embodiment of the present invention;

FIG. 13 is a block diagram showing the structure of part of the photoacoustic imager according to the fourth embodiment of the present invention;

FIG. 14 is a timing chart for illustrating operations of the photoacoustic imager according to the fourth embodiment of the present invention;

FIG. 15 is a circuit diagram showing the structure of a current detection portion according to a first modification of the first embodiment of the present invention;

FIG. 16 is a block diagram showing the structure of part of a photoacoustic imager according to a second modification of the first embodiment of the present invention;

FIG. 17 is a block diagram showing the structure of part of a photoacoustic imager according to a third modification of the first embodiment (the third embodiment) of the present invention; and

FIG. 18 is a block diagram showing the structure of a light-emitting element group according to each of fourth and fifth modifications of the first embodiment of the present invention.

### DESCRIPTION OF THE PREFERRED EMBODIMENTS

Embodiments of the present invention are now described with reference to the drawings.

### First Embodiment

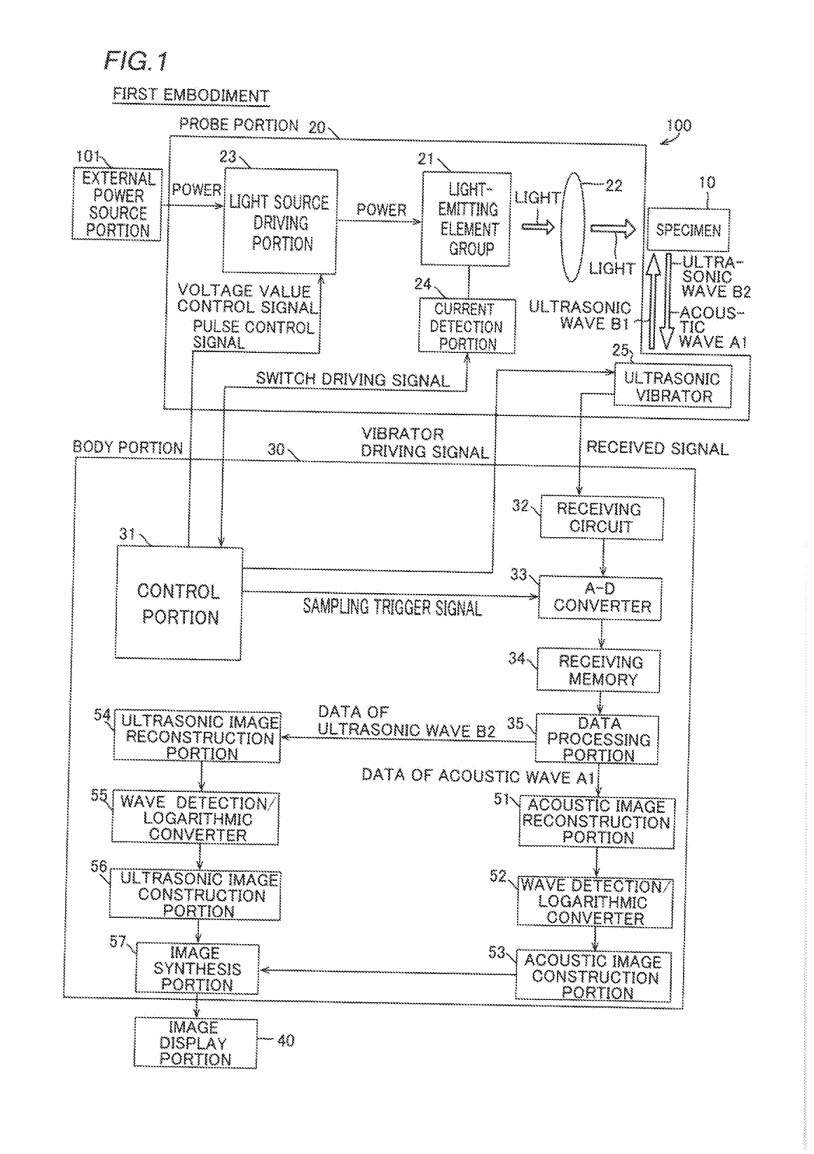

The structure of a photoacoustic imager 100 according to a first embodiment of the present invention is described with reference to FIGS. 1 to 3.

The photoacoustic imager 100 according to the first embodiment of the present invention is provided with a probe portion 20, as shown in FIG. 1. The probe portion 20 is configured to detect an acoustic wave A1 and an ultrasonic wave B2 from a specimen 10 and to transmit the same to a body portion 30 described later as received signals.

The photoacoustic imager 100 is also provided with the body portion 30. The body portion 30 is configured to process and image the received signals detected by the probe portion 20.

The photoacoustic imager 10 is also provided with an image display portion 40. The image display portion 40 is configured to be capable of acquiring and displaying images processed by the body portion 30.

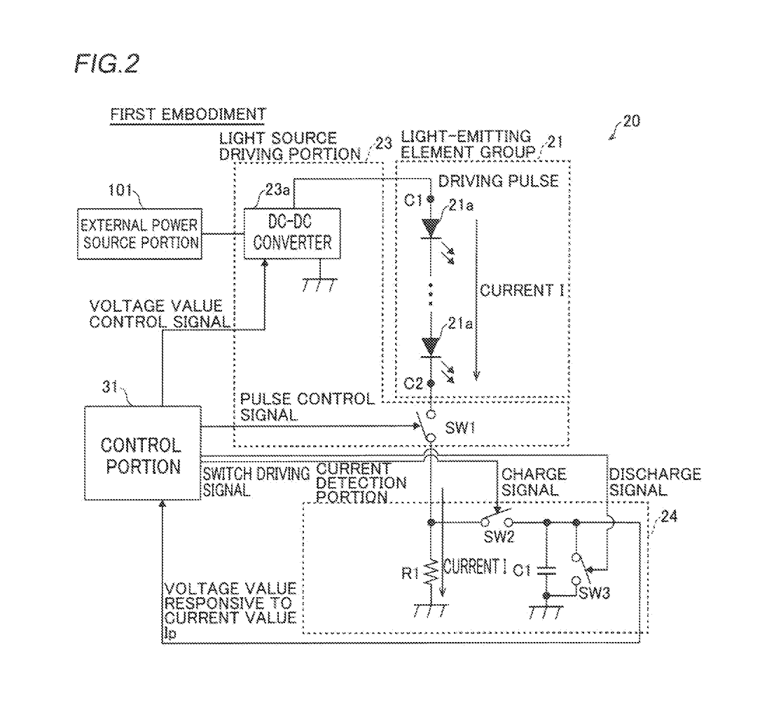

The probe portion 20 is provided with a light-emitting element group 21. The light-emitting element group 21 includes a plurality of light-emitting diode elements 21a (see FIG. 2) capable of emitting pulsed light having a wavelength (a wavelength of about 600 nm to 1000 nm, for example, and preferably about 850 nm) in the infrared region.

The probe portion 20 is also provided with a condensing lens 22. The condensing lens 22 is configured to apply the pulsed light from the light-emitting element group 21 to the specimen 10 while condensing the same.

A detection object (hemoglobin or the like, for example) in the specimen 10 absorbs the pulsed light applied from the probe portion 20 to the specimen 10. The detection object (the specimen 10) generates the acoustic wave A1 by expanding and contracting (returning to the original size from an expanding state) in response to the intensity of application (the quantity of absorption) of the pulsed light. Throughout the specification, the “acoustic wave A1” denotes an ultrasonic wave generated by the detection object in the specimen 10 absorbing light, and the “ultrasonic wave B2” denotes an ultrasonic wave generated by an ultrasonic vibrator 25 and reflected by the specimen 10, for the convenience of illustration.

The probe portion 20 is also provided with a light source driving portion 23. The light source driving portion 23 is configured to acquire power from an external power source portion 101. Further, the light source driving portion 23 is configured to supply the power to the light-emitting element group 21 on the basis of a voltage value control signal and a pulse control signal received from a control portion 31 described later.

The probe portion 20 is also provided with a current detection portion 24. The current detection portion 24 is configured to be capable of detecting the value Ip of current I flowing in the plurality of light-emitting diode elements 21a of the light-emitting element group 21.

The probe portion 20 is also provided with the ultrasonic vibrator 25. The ultrasonic vibrator 25 is constituted of a piezoelectric element (lead zirconate titanate (PZT), for example). The ultrasonic vibrator 25 is configured to vibrate and generate voltage (received signal) when acquiring the aforementioned acoustic wave A1. Further, the ultrasonic vibrator 25 is configured to transmit the acquired received signal to a receiving circuit 32 described later. The ultrasonic vibrator 25 is an example of the “acoustic wave detection portion” in the present invention.

The ultrasonic vibrator 25 is further configured to be capable of generating an ultrasonic wave B1 by vibrating at a frequency responsive to a vibrator driving signal received from the control portion 31. A substance, having high acoustic impedance, in the specimen 10 reflects the ultrasonic wave B1 generated by the ultrasonic vibrator 25. The ultrasonic vibrator 25 is further configured to acquire the ultrasonic wave B2 (resulting from reflection of the ultrasonic wave B1) and to vibrate due to the ultrasonic wave B2.

The ultrasonic vibrator 25 is further configured to transmit a received signal to the receiving circuit 32 also in the case of vibrating due to the ultrasonic wave B2, similarly to the case of vibrating due to the acoustic wave A1. The photoacoustic imager 100 is configured not to superpose a period for applying the pulsed light to the specimen 10 with the light-emitting element group 21 so that the specimen 10 generates the acoustic wave A1 and the ultrasonic vibrator 25 acquires the acoustic wave A1 and a period for applying the ultrasonic wave B1 to the specimen 10 with the ultrasonic vibrator 25 so that the ultrasonic vibrator 25 acquires the ultrasonic wave B2, to be capable of distinguishing the acoustic wave A1 and the ultrasonic wave B2 from each other.

The body portion 30 is provided with the control portion 31, as shown in FIG. 1. The control portion 31 includes a CPU (central processing unit) and the like, and is configured to control the entire photoacoustic imager 100 by transmitting control signals to respective portions.

The body portion 30 is also provided with the receiving circuit 32. The receiving circuit 32 includes a coupling capacitor or the like, and is configured to acquire the received signal (alternating component) from the ultrasonic vibrator 25. The receiving circuit 32 is further configured to transmit the acquired received signal to an A-D converter 33.

The body portion 30 is also provided with the A-D converter 33. The A-D converter 33 is configured to convert the received signal (analog signal) acquired from the receiving circuit 32 to a digital signal in correspondence to a sampling trigger signal acquired from the control portion 31. The A-D converter 33 is connected with a receiving memory 34, and configured to transmit the received signal converted to the digital signal to the receiving memory 34.

The body portion 30 is also provided with the receiving memory 34. The receiving memory 34 is configured to temporarily store the received signal converted to the digital signal. Further, the receiving memory 34 is connected with a data processing portion 35, and configured to transmit a stored acoustic wave signal to the data processing portion 35.

The body portion 30 is also provided with the data processing portion 35. The data processing portion 35 is connected with an acoustic image reconstruction portion 51, and configured to transmit data of the acoustic wave A1 to the acoustic image reconstruction portion 51. The data processing portion 35 is also connected with an ultrasonic image reconstruction portion 54, and configured to transmit data of the ultrasonic wave B2 to the ultrasonic image reconstruction portion 54.

The body portion 30 is also provided with the acoustic image reconstruction portion 51. The acoustic image reconstruction portion 51 is configured to perform processing of reconstructing the acquired data of the acoustic wave A1 as an image. The acoustic image reconstruction portion 51 is connected with a wave detection/logarithmic converter 52, and configured to transmit the data of the acoustic wave A1 reconstructed as an image to the wave detection/logarithmic converter 52.

The body portion 30 is also provided with the wave detection/logarithmic converter 52. The wave detection/logarithmic converter 52 is configured to perform waveform processing of data reconstructed as an image. Further, the wave detection/logarithmic converter 52 is connected with an acoustic image construction portion 53, and configured to transmit the waveform-processed data.

The body portion 30 is also provided with the acoustic image construction portion 53. The acoustic image construction portion 53 is configured to perform processing of constructing a tomographic image in the specimen 10 on the basis of the waveform-processed data. Further, the acoustic image construction portion 53 is connected with an image synthesis portion 57, and configured to transmit a tomographic image based on the acoustic wave A1.

The body portion 30 is also provided with the ultrasonic image reconstruction portion 54, a wave detection/logarithmic converter 55, an ultrasonic image construction portion 56 and the image synthesis portion 57. The ultrasonic image reconstruction portion 54 is configured to perform processing of reconstructing the data of the ultrasonic wave B2 acquired from the data processing portion 35 as an image. Further, the ultrasonic image reconstruction portion 54 is configured to transmit a tomographic image based on the ultrasonic wave B2 to the image synthesis portion 57 through the wave detection/logarithmic converter 55 and the ultrasonic image construction portion 56.

The image synthesis portion 57 is configured to perform processing of synthesizing the tomographic images based on the acoustic wave A1 and the ultrasonic wave B2 and to output a synthetic image to the image display portion 40.

The image display portion 40 is constituted of a liquid crystal panel or the like, and configured to display an image acquired from the body portion 30.

The light source driving portion 23 is provided with a DC-DC converter 23a, as shown in FIG. 2. The DC-DC converter 23a is configured to convert power acquired from the external power source portion 101 to a voltage value based on the voltage value control signal acquired from the control portion 31 and to apply the converted voltage to the light-emitting diode elements 21a of the light-emitting element group 21.

The light-emitting element group 21 is provided with the plurality of light-emitting diode elements 21a serially connected with each other. The DC-DC converter 23a applies the voltage to anode sides (points C1) of the light-emitting diode elements 21a. Cathode sides (points C2) of the light-emitting diode elements 21a are connected with a switch portion SW1.

The light source driving portion 23 is also provided with the switch portion SW1. The switch portion SW1 is configured to be capable of switching conduction and disconnection of the light-emitting element group 21 and the current detection portion 24 on the basis of the pulse control signal received from the control portion 31.

Thus, potential difference is caused between the points C1 and C2 when the switch portion SW1 is in an ON-state (conducting state), whereby the current I flows in the plurality of light-emitting diode elements 21a, which in turn emit light. In other words, the light source driving portion 23 (the DC-DC converter 23a) supplies power to the light-emitting element group 21 in this case. When the switch portion SW1 is in an OFF-state (disconnected state), on the other hand, no current flows in the plurality of light-emitting diode elements 21a. In other words, the light source driving portion 23 (the DC-DC converter 23a) stops supplying power to the light-emitting element group 21 in this case.

The control portion 31 is configured to transmit the pulse control signal (see FIG. 5) for repeatedly turning on/off the switch portion SW1. Thus, the photoacoustic imager 100 is so configured that the potential difference between the points C1 and C2 of the light-emitting element group 21 has a driving pulse (driving voltage) in the form of a rectangular wave. The pulse control signal is so set that an ON-period is 150 nm and a repetition frequency is 1 kHz or the like, and set to such a value that the light-emitting diode elements 21a can efficiently emit light with small heat generation.

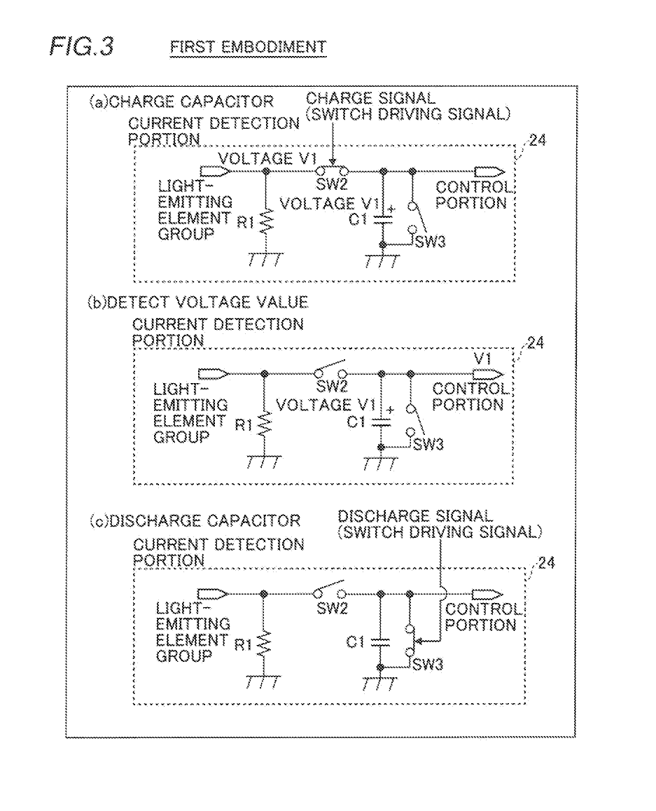

As shown in FIG. 3, the current detection portion 24 includes a peak holding circuit, and is configured to be capable of detecting the value Ip of the current I flowing in the light-emitting element group 21. The current detection portion 24 is now described in more detail.

The current detection portion 24 is provided with a detection resistor R1, a charge capacitor C1, a charge switch portion SW2 and a discharge switch portion SW3. A first end of the detection resistor R1 is connected with the aforementioned switch portion SW1 and the charge switch portion SW2, while a second end thereof is grounded. Thus, the detection resistor R1 is configured to be capable of applying voltage V1 responsive to the current value Ip to the charge switch portion SW2. The current value Ip (the value of the current I flowing in the light-emitting element group 21) has the following relation (1) with the detection resistor R1 and the voltage V1. The charge switch portion SW2 and the discharge switch portion SW3 are examples of the “detection switch portion” in the present invention.

Ip=V1/R1 (1)

As shown at (a) in FIG. 3, the charge switch portion SW2 is configured to be capable of switching ON- and OFF-states in response to a switch driving signal (a charge signal) received from the control portion 31. Further, the charge switch portion SW2 is configured to charge the charge capacitor C1 by applying the voltage V1 applied to the detection resistor R1 to the charge capacitor C1 by being turned on.

When the charge switch portion SW2 is turned off, the charge capacitor C1 maintains the voltage V1, as shown at (b) in FIG. 3. In other words, the control portion 31 is configured to be capable of acquiring the voltage V1 and acquiring the current value Ip on the basis of the acquired voltage V1 after turning off the charge switch portion SW2.

The discharge switch portion SW3 is configured to acquire a switch driving signal (a discharge signal) from the control portion 31 so that the charged charge capacitor C1 discharges when the discharge switch portion SW3 is turned on, as shown at (c) in FIG. 3. Thus, the charge capacitor C1 returns to an uncharged state.

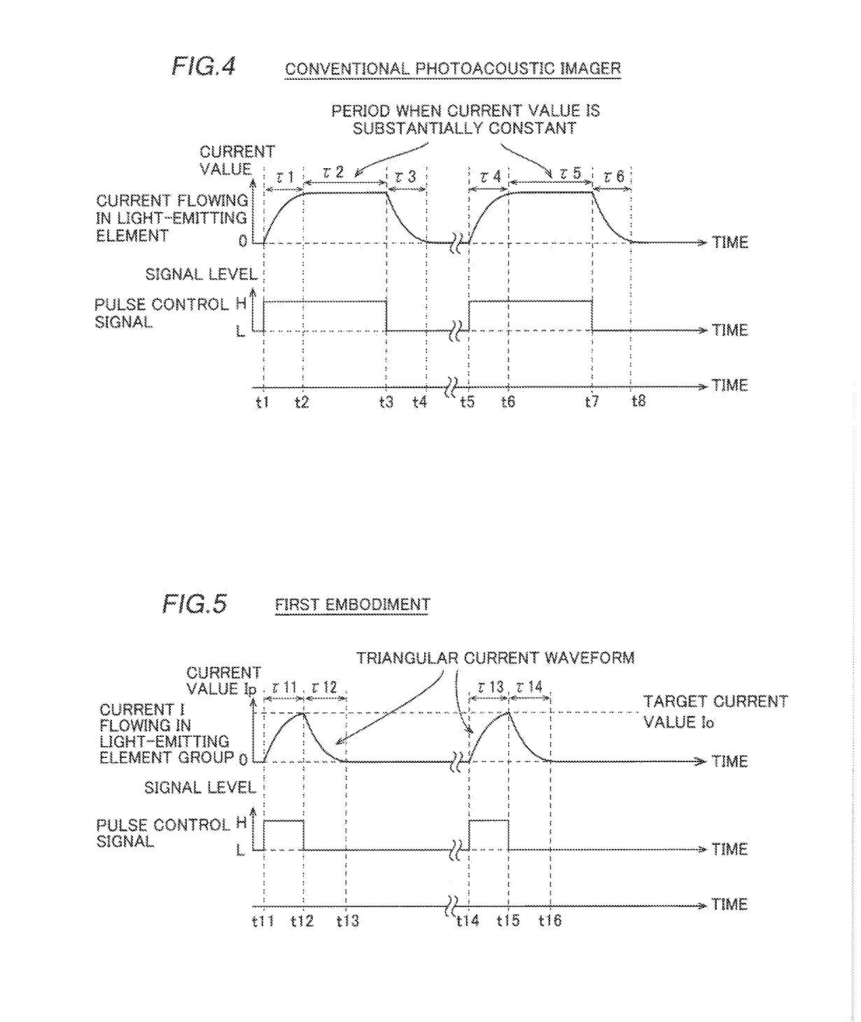

A driving method of a light source driving portion in a conventional photoacoustic imager is now described with reference to FIG. 4.

The conventional photoacoustic imager has periods (τ2 and τ5) when the value of current flowing in a light-emitting element becomes substantially constant. The value of the current flowing in the light-emitting element and the magnitude of light (pulsed light) emitted from the light-emitting element correspond to each other, and hence this indicates that the conventional photoacoustic imager has the periods (τ2 and τ5) when the magnitude of the light emitted from the light-emitting element becomes substantially constant.

More specifically, when a pulse control signal is set to a high level at times t1 to t3, the value of the current flowing in the light-emitting element rises at the times t1 to t2, and becomes substantially constant at the times t2 to t3. Further, the value of the current flowing in the light-emitting element gradually decreases at times t3 to t4. At times t5 to t8, the value of the current flowing in the light-emitting element has a waveform similar to that at the times t1 to t4.

In periods (τ1, τ3, τ4 and τ6) when the magnitude of light absorbed by a detection object in a specimen changes, the detection object generates an acoustic wave. In the periods (τ2 and τ5) when the magnitude of light absorbed by the detection object in the specimen remains unchanged, the detection object generates no acoustic wave.

A driving method of the light source driving portion 23 in the photoacoustic imager 100 according to the first embodiment is now described with reference to FIG. 5.

According to the first embodiment, the light source driving portion 23 starts supplying power to the light-emitting element group 21 in a state where the value Ip of the current I flowing in the light-emitting element group 21 is substantially zero, and stops supplying power to the light-emitting element group 21 on the basis of that the value Ip of the current I flowing in the light-emitting element group 21 has reached a target current value Io so that the waveform of the current I flowing in the light-emitting element group 21 becomes triangular. Thus, the light-emitting diode elements 21a emit pulsed light having a triangular waveform according to the first embodiment. This is now specifically described.

The pulse control signal is set to a high level at a time t11 (a state where the value Ip of the current I flowing in the light-emitting element group 21 is substantially zero), and the value Ip of the current I flowing in the light-emitting element group 21 gradually increases. Then, the value Ip of the current I flowing in the light-emitting element group 21 reaches the target current value Io at a time t12. Then, the pulse control signal is set to a low level on the basis of that the value Ip of the current I flowing in the light-emitting element group 21 has reached the target current value Io. The target current value Io is an example of the “prescribed current value” in the present invention.

The pulse control signal is so set to a low level that the value Ip of the current I flowing in the light-emitting element group 21 gradually decreases at times t12 to t13. At times t14 to t16, the current I flowing in the light-emitting element group 21 has a waveform similar to that at the times t11 to t13.

Therefore, the waveform of the current I flowing in the light-emitting element group 21 becomes triangular, and there is no period when the value Ip of the current I flowing in the light-emitting element group 21 becomes substantially constant. Thus, the detection object generates the acoustic wave A1 in periods (τ11, τ12, τ13 and τ14) when the magnitude of light absorbed by the detection object in the specimen 10 changes. In other words, the detection object generates the acoustic wave A1 substantially in all periods when the current I flows in the light-emitting element group 21. The periods 11, τ12, τ13 and τ14 are examples of the “periods for feeding the current to the light-emitting element” in the present invention. The periods τ11 and τ13 are examples of the “first period” in the present invention. The periods τ12 and τ14 are examples of the “second period” in the present invention. According to the first embodiment, the first periods (τ11 and τ13) correspond to rise times of the light-emitting diode elements 21a, while the second periods (τ12 and τ14) correspond to fall times of the light-emitting diode elements 21a.

According to the first embodiment, the following effects can be attained:

According to the first embodiment, as hereinabove described, the light source driving portion 23 is configured to stop supplying power to the light-emitting element group 21 on the basis of that the value Ip of the current I flowing in the light-emitting element group 21 has reached the target current value Io. Thus, the light source driving portion 23 can stop supplying power to the light-emitting element group 21 before the value Ip of the current I flowing in the light-emitting element group 21 reaches a substantially unchanged current value (the target current value Io), whereby a period when the magnitude of the value Ip of the current I flowing in the light-emitting element group 21 remains unchanged can be shortened or eliminated. The photoacoustic imager 100 can prevent emission of light not contributing to generation of the acoustic wave A1 by shortening or eliminating the period when the magnitude of the value Ip of the current I flowing in the light-emitting element group 21 remains unchanged. Consequently, the photoacoustic imager 100 can prevent increase in power consumption for emitting light by preventing emission of light not contributing to generation of the acoustic wave A1. Further, the photoacoustic imager 100 can also prevent heat generation resulting from power consumption by preventing increase in power consumption.

According to the first embodiment, as hereinabove described, the light source driving portion 23 is configured to start supplying power to the light-emitting element group 21 in the state where the value Ip of the current I flowing in the light-emitting element group 21 is substantially zero and to stop supplying power to the light-emitting element group 21 on the basis of that the value Ip of the current I flowing in the light-emitting element group 21 has reached the target current value Io, so that the waveform of the current I flowing in the light-emitting element group 21 becomes triangular. Thus, the photoacoustic imager 100 can further shorten the period when the magnitude of the value Ip of the current I flowing in the light-emitting element group 21 remains unchanged as compared with a case of making the waveform of the current I flowing in the light-emitting element group 21 rectangular, by making the waveform of the current I triangular. Consequently, the photoacoustic imager 100 can further prevent increase in power consumption for emitting light by preventing emission of light not contributing to generation of the acoustic wave A1.

According to the first embodiment, as hereinabove described, the light-emitting element group 21 is configured to include the light-emitting diode elements 21a. The light-emitting diode elements 21a are lower in directivity as compared with light-emitting elements emitting laser beams, and a light emission range remains relatively unchanged also when misregistration takes place. Therefore, the photoacoustic imager 100 requires neither precise alignment (registration) of optical members nor an optical platen or a strong housing for preventing characteristic fluctuation resulting from vibration of an optical system. Consequently, the photoacoustic imager 100 can be prevented from size increase and complication in structure due to the nonrequirement for precise alignment of optical members and an optical platen or a strong housing.

According to the first embodiment, as hereinabove described, the light source driving portion 23 is configured to change the value Ip of the current I flowing in the light-emitting diode elements 21a substantially in all periods (τ11 to τ14) for feeding the current I to the light-emitting diode elements 21a. Thus, a period when the magnitude of the current I flowing in the light-emitting diode elements 21a remains unchanged is substantially eliminated from the periods for feeding the current I to the light-emitting diode elements 21a, whereby the photoacoustic imager 100 can further prevent emission of light not contributing to generation of the acoustic wave A1.

According to the first embodiment, as hereinabove described, the periods (τ11 to τ14) for feeding the current I to the light-emitting diode elements 21a consist of the first periods (τ11 and τ13) when the value Ip of the current I flowing in the light-emitting diode elements 21a increases from the substantially zero state and the second periods (τ12 and τ14) when the value Ip of the current I flowing in the light-emitting diode elements 21a decreases from the target current value Io. Thus, the value Ip of the current I flowing in the light-emitting diode elements 21a can be easily changed in substantially all periods for feeding the current I to the light-emitting diode elements 21a.

According to the first embodiment, as hereinabove described, the first periods (τ11 and τ13) correspond to the rise times of the light-emitting diode elements 21a, while the second periods (τ12 and τ14) correspond to the fall times of the light-emitting diode elements 21a. Thus, the first periods when the value Ip of the current I flowing in the light-emitting diode elements 21a increases from the substantially zero state and the second periods when the value Ip of the current I flowing in the light-emitting diode elements 21a decreases from the target current value Io can be easily set by making the first periods correspond to the rise times of the light-emitting diode elements 21a and making the second periods correspond to the fall times of the light-emitting diode elements 21a.

According to the first embodiment, as hereinabove described, the light-emitting diode elements 21a are configured to emit pulsed light having a triangular waveform by being supplied with power from the light source driving portion 23. Thus, the pulsed light having a triangular waveform has no period when the intensity of light remains unchanged (the intensity of the light becomes substantially constant), whereby the photoacoustic imager 100 can prevent emission of light not contributing to generation of the acoustic wave A1. Consequently, the photoacoustic imager 100 can prevent increase in power consumption for emitting light.

According to the first embodiment, as hereinabove described, the light-emitting diode elements 21a are configured to emit pulsed light having a wavelength in the infrared region. Thus, the light having the wavelength in the infrared region can relatively easily penetrate a human body, whereby the photoacoustic imager 100 can deliver the light from the light-emitting diode elements 21a to a deeper portion of the specimen 10 when the specimen 10 is prepared from a human body.

According to the first embodiment, as hereinabove described, the photoacoustic imager 100 is provided with the current detection portion 24 detecting the value Ip of the current I flowing in the light-emitting diode elements 21a, and the current detection portion 24 is configured to include the detection resistor R1, the charge capacitor C1, the charge switch portion SW2 and the discharge switch portion SW3. Thus, the detection resistor R1, the charge capacitor C1, the charge switch portion SW2 and the discharge switch portion SW3 can constitute a peak holding circuit, whereby the photoacoustic imager 100 can easily acquire the value Ip (peak value) of the current I flowing in the light-emitting diode elements 21a with the peak holding circuit.

### Second Embodiment

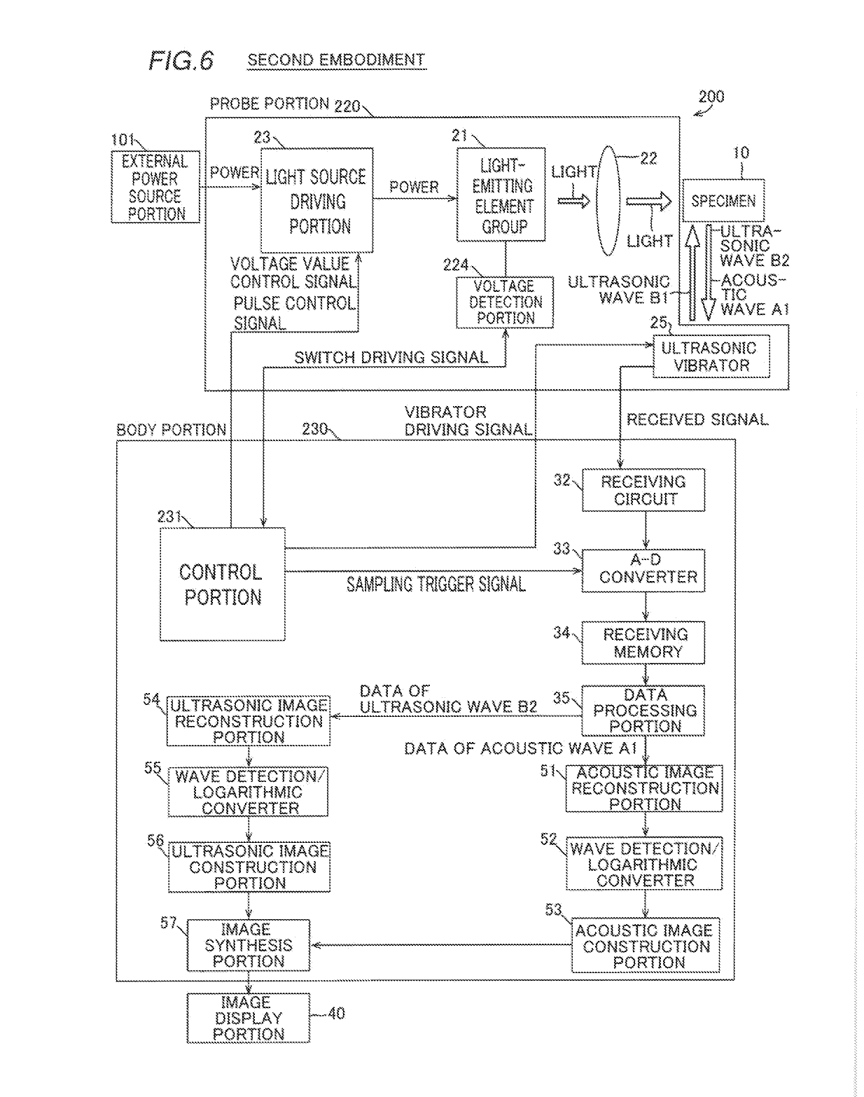

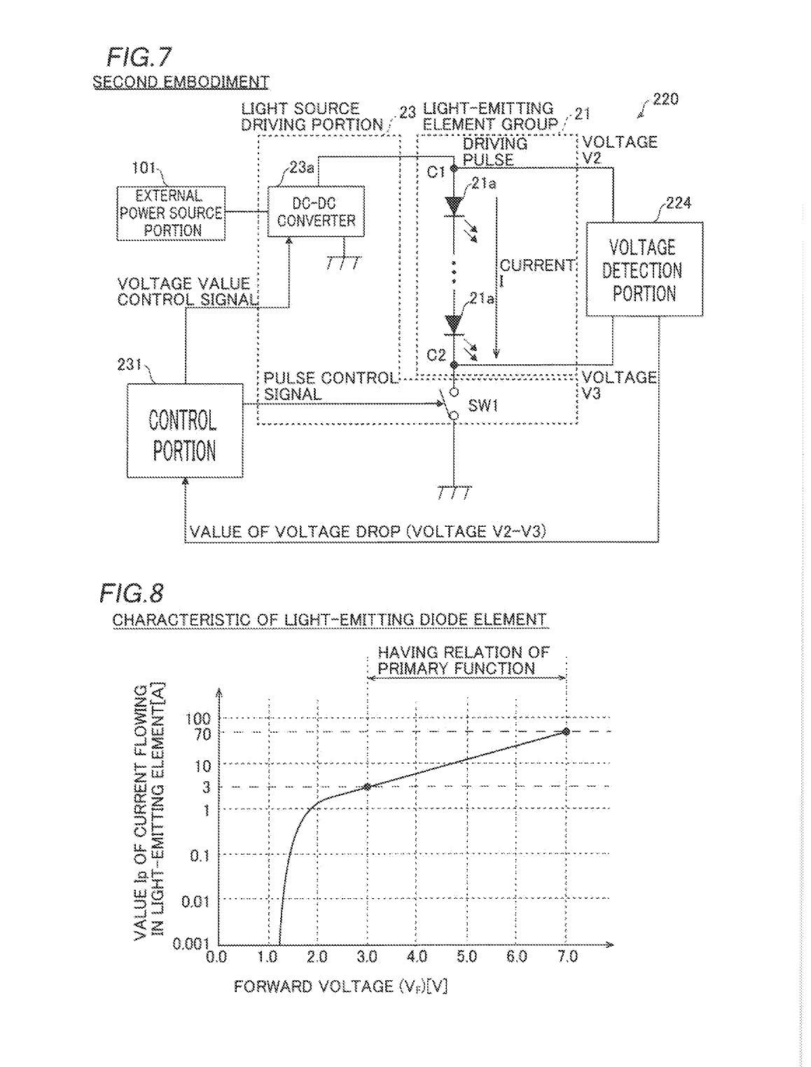

The structure of a photoacoustic imager 200 according to a second embodiment of the present invention is now described with reference to FIGS. 6 to 8. According to the second embodiment, the photoacoustic imager 200 is provided with a voltage detection portion 224 configured to be capable of detecting the magnitude of a voltage drop caused by a light-emitting element group 21 in voltage V2 applied from a light source driving portion 23, dissimilarly to the photoacoustic imager 100 according to the first embodiment provided with the current detection portion 24 configured to be capable of detecting the peak of the value Ip of the current I flowing in the light-emitting diode elements 21a.

As shown in FIG. 6, the photoacoustic imager 200 according to the second embodiment is provided with a probe portion 220.

The photoacoustic imager 200 is also provided with a body portion 230. The body portion 230 includes a control portion 231.

According to the second embodiment, the probe portion 220 is provided with the voltage detection portion 224 acquiring the value Ip of the current I flowing in the light-emitting element group 21 by detecting the magnitude (voltage V2−voltage V3) of a voltage drop in the voltage V2 applied to the light-emitting element group 21 upon power supply from the light source driving portion 23. The voltage detection portion 224 is an example of the “current detection portion” in the present invention.

More specifically, the voltage detection portion 224 is configured to be capable of detecting the potential difference between voltage values V2 and V3 on points C1 and C2 of the light-emitting element group 21. Further, the voltage detection portion 224 is configured to transmit information of the detected potential difference (the magnitude of the voltage drop) to the control portion 231.

As shown in FIG. 8, the control portion 231 is configured to be capable of calculating the value Ip of the current I flowing in the light-emitting element group 21 on the basis of the acquired magnitude of the voltage drop. For example, the value of a voltage drop per light-emitting diode element 21a in a plurality of light-emitting diode elements 21a included in the light-emitting element group 21 coincides with the value of forward voltage V_{F}. Thus, the control portion 231 calculates the value Ip of the current I flowing in the light-emitting element group 21 from the acquired magnitude of the voltage drop on the basis of characteristics of the light-emitting diode elements 21a shown in FIG. 8. For example, the value Ip of the current I flowing in the light-emitting element group 21 is 3 A when the forward voltage V_{F} is 3 V, and 70 A when the forward voltage V_{F} is 7 V. The characteristics of the light-emitting diode elements 21a are examples of the “current characteristic of the light-emitting element corresponding to the magnitude of the voltage drop in the voltage” in the present invention.

The forward voltage V_{F} of the light-emitting diode elements 21a has a property of becoming a primary function with the value Ip of the current I flowing in the light-emitting element group 21 expressed as a logarithm in a voltage range where the forward voltage V_{F} is relatively large (a range where the forward voltage V_{F} is at least 3 V and not more than 7 V, for example). Thus, the control portion 231 can prevent increase in a burden on processing of the control portion 231 by calculating the value Ip of the current I flowing in the light-emitting element group 21 in consideration of the property of the forward voltage V_{F} becoming a primary function.

The control portion 231 is also configured to substantially null the value Ip of the current I flowing in the light-emitting element group 21 by stopping supplying power to the light-emitting element group 21 on the basis of that the value Ip of the current I flowing in the light-emitting element group 21 has reached a target current value Io similarly to the control portion 31 of the photoacoustic imager 100 according to the first embodiment, by controlling driving of the light source driving portion 23 on the basis of the calculated value Ip of the current I flowing in the light-emitting element group 21. Thus, the photoacoustic imager 200 according to the second embodiment can also shorten periods when the magnitude of the value Ip of the current I flowing in the light-emitting element group 21 remains unchanged, similarly to the photoacoustic imager 100 according to the first embodiment.

The remaining structures of the photoacoustic imager 200 according to the second embodiment are similar to those of the photoacoustic imager 100 according to the first embodiment.

According to the second embodiment, the following effects can be attained:

According to the second embodiment, as hereinabove described, the photoacoustic imager 200 is provided with the voltage detection portion 224 acquiring the value Ip of the current I flowing in the light-emitting element group 21 by detecting the magnitude of the voltage drop in the voltage V2 applied thereto upon power supply from the light source driving portion 23. Generally in the case of detecting the value Ip of the current I flowing in the light-emitting element group 21, the current detection portion 24 (the peak holding circuit) (see FIG. 2) must be provided, as in the photoacoustic imager 100 according to the first embodiment, for example. On the other hand, the value Ip of the current I flowing in the light-emitting element group 21 is correlated with the magnitude (the forward voltage V_{F}) of the voltage drop in the voltage V2 applied to the light-emitting element group 21. When the voltage detection portion 224 is configured to acquire the value Ip of the current I flowing in the light-emitting element group 21 by detecting the magnitude of the voltage drop in the voltage V2 applied thereto as hereinabove described, the value Ip of the current I flowing in the light-emitting element group 21 can be acquired without providing the current detection portion 24 (the peak holding circuit) or the like. The structure of the voltage detection portion 224 is not complicated as compared with the current detection portion 24, and hence the photoacoustic imager 200 can be prevented from complication in structure.

According to the second embodiment, as hereinabove described, the control portion 231 is configured to acquire the value Ip of the current I flowing in the light-emitting diode elements 21a on the basis of the magnitude of the voltage drop in the voltage V2 and the current characteristics of the light-emitting diode elements 21a corresponding to the magnitude of the voltage drop in the voltage V2. Thus, the voltage detection portion 224 so detects the magnitude of the voltage drop in the voltage V2 that the control portion 231 can easily calculate the value Ip of the current I flowing in the light-emitting diode elements 21a by employing the current characteristics of the light-emitting diode elements 21a corresponding to the magnitude of the voltage drop in the voltage V2.

According to the second embodiment, as hereinabove described, the current characteristics of the light-emitting diode elements 21a corresponding to the magnitude of the voltage drop in the voltage V2 are those associating the forward voltage V_{F} of the light-emitting diode elements 21a and the value Ip of the current I flowing in the light-emitting diode elements 21a with each other. It is generally known that the forward voltage V_{F} of the light-emitting diode elements 21a and the value Ip of the current I flowing in the light-emitting diode elements 21a are correlated with each other. Further, the magnitude of the voltage drop per light-emitting diode element 21a and the forward voltage V_{F} of the light-emitting diode elements 21a substantially coincide with each other. Noting these points, the photoacoustic imager 200 according to the second embodiment can more easily acquire the value Ip of the current I flowing in the light-emitting diode elements 21a by employing the magnitude of the voltage drop in the voltage V2 and the characteristics associating the forward voltage V_{F} of the light-emitting diode elements 21a and the value Ip of the current I flowing in the light-emitting diode elements 21a with each other.

The remaining effects of the photoacoustic imager 200 according to the second embodiment are similar to those of the photoacoustic imager 100 according to the first embodiment.

### Third Embodiment

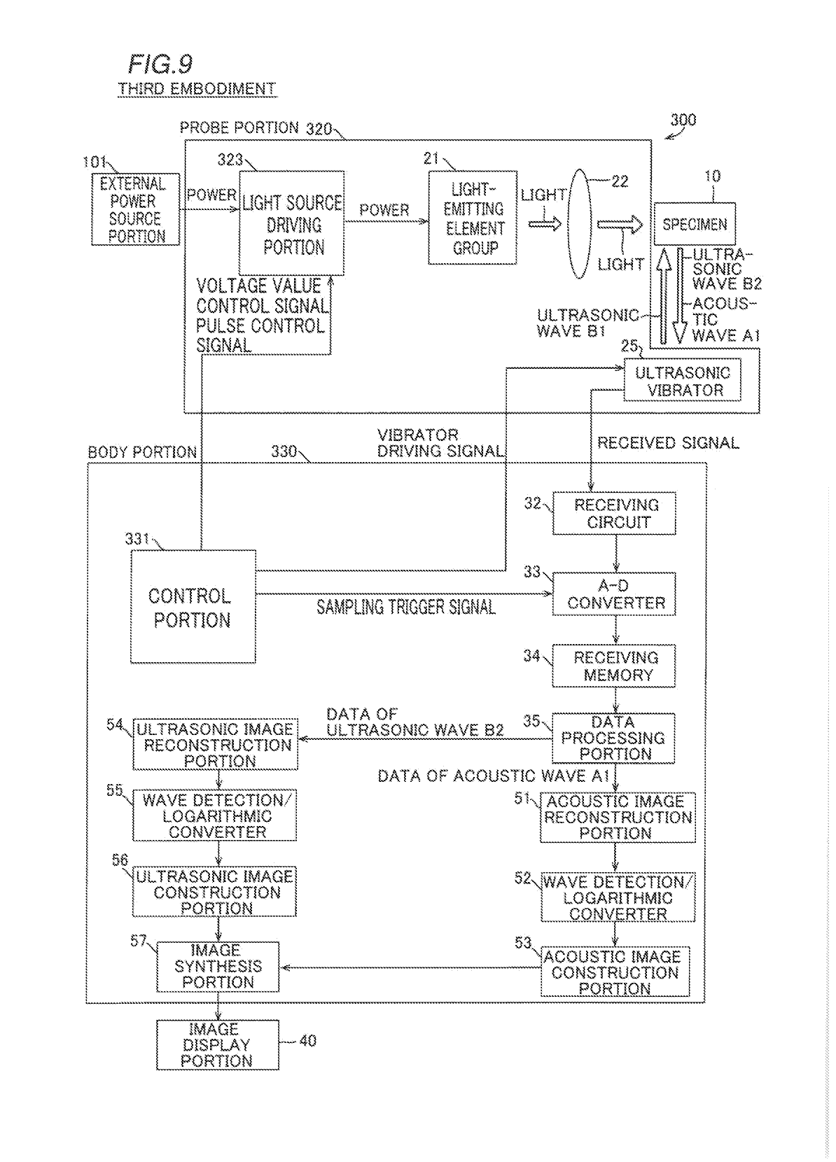

The structure of a photoacoustic imager 300 according to a third embodiment of the present invention is now described with reference to FIGS. 9 to 11. According to the third embodiment, the photoacoustic imager 300 is configured to control driving of a light source driving portion 323 on the basis of a table 331a including a voltage value V4 corresponding to a target current value Io and a pulse width tw also corresponding to the target current value Io, dissimilarly to the photoacoustic imager 100 according to the first embodiment configured to acquire the value Ip of the current I flowing in the light-emitting element group 21 and to control driving of the light source driving portion 23 on the basis of the acquired value Ip of the current I flowing in the light source element group 21.

As shown in FIG. 9, the photoacoustic imager 300 according to the third embodiment is provided with a probe portion 320.

The photoacoustic imager 300 is also provided with a body portion 330. The body portion 330 includes a control portion 331.

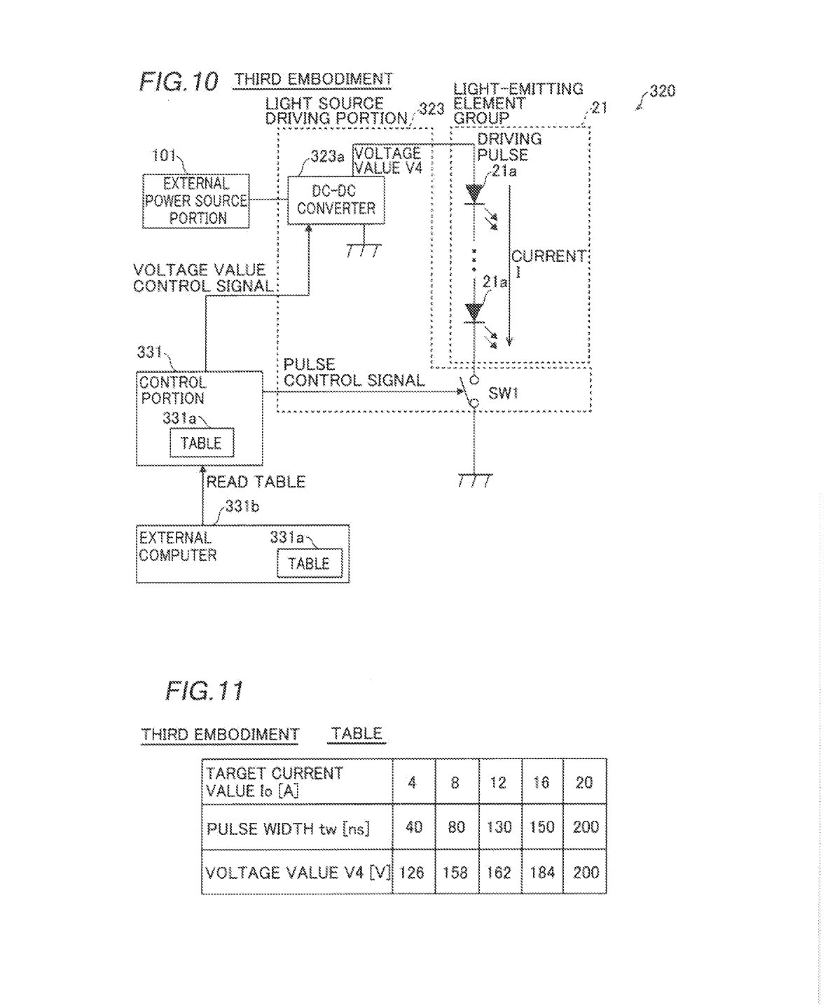

According to the third embodiment, the light source driving portion 323 is configured to supply a driving pulse based on the table 331a including the voltage value V4 corresponding to the target current value Io and the pulse width tw also corresponding to the target current value Io to a light-emitting element group 21, as shown in FIGS. 10 and 11.

More specifically, the control portion 331 is provided with the table 331a, as shown in FIG. 10. The table 331a includes information about pulse widths tw and voltage values V4 corresponding to a plurality of different target current values Io, as shown in FIG. 11. In a case of driving the light source driving portion 323 so that the target current value Io becomes 12 A, for example, the control portion 331 so drives the light source driving portion 323 that the pulse width tw becomes 130 ns and the voltage value V4 becomes 162 V, whereby current I flowing in the light-emitting element group 21 has such a triangular waveform that a current value Ip reaches 12 A (a peak value). Thus, the photoacoustic imager 300 according to the third embodiment can also shorten periods when the value Ip of the current I flowing in the light-emitting element group 21 remains unchanged, similarly to the photoacoustic imager 100 according to the first embodiment.

The control portion 331 is configured to be connectable with an external computer 331b, and to be capable of acquiring the table 331a from the external computer 331b. When the light-emitting element group 21 is exchanged, therefore, the control portion 331 can acquire the table 331a corresponding to the exchanged light-emitting element group 21. When emission wavelengths of light-emitting diode elements 21a included in the unexchanged and exchanged light-emitting element groups 21 are different from each other or production lots are different from each other while the emission wavelengths are identical to each other, for example, the photoacoustic imager 300 can more reliably shorten the periods when the magnitude of the value Ip of the current I flowing in the light-emitting element group 21 remains unchanged. The remaining structures of the photoacoustic imager 300 according to the third embodiment are similar to those of the photoacoustic imager 100 according to the first embodiment.

According to the third embodiment, the following effect can be attained:

According to the third embodiment, as hereinabove described, the light source driving portion 323 is configured to supply the driving pulse based on the table 331a including the voltage value V4 corresponding to the target current value Io and the pulse width tw also corresponding to the target current value Io to the light-emitting element group 21. Thus, the light source driving portion 323 can stop supplying power to the light-emitting element group 21 when the value Ip of the current I flowing in the light-emitting element group 21 reaches the target current value Io by supplying the driving pulse based on the table 331a to the light-emitting element group 21, without acquiring the value Ip of the current I flowing in the light-emitting element group 21. Consequently, the photoacoustic imager 300 may be provided with no current detection portion for acquiring the value Ip of the current I flowing in the light-emitting element group 21, whereby the same can be further prevented from complication in structure.

The remaining effects of the photoacoustic imager 300 according to the third embodiment are similar to those of the photoacoustic imager 100 according to the first embodiment.

### Fourth Embodiment

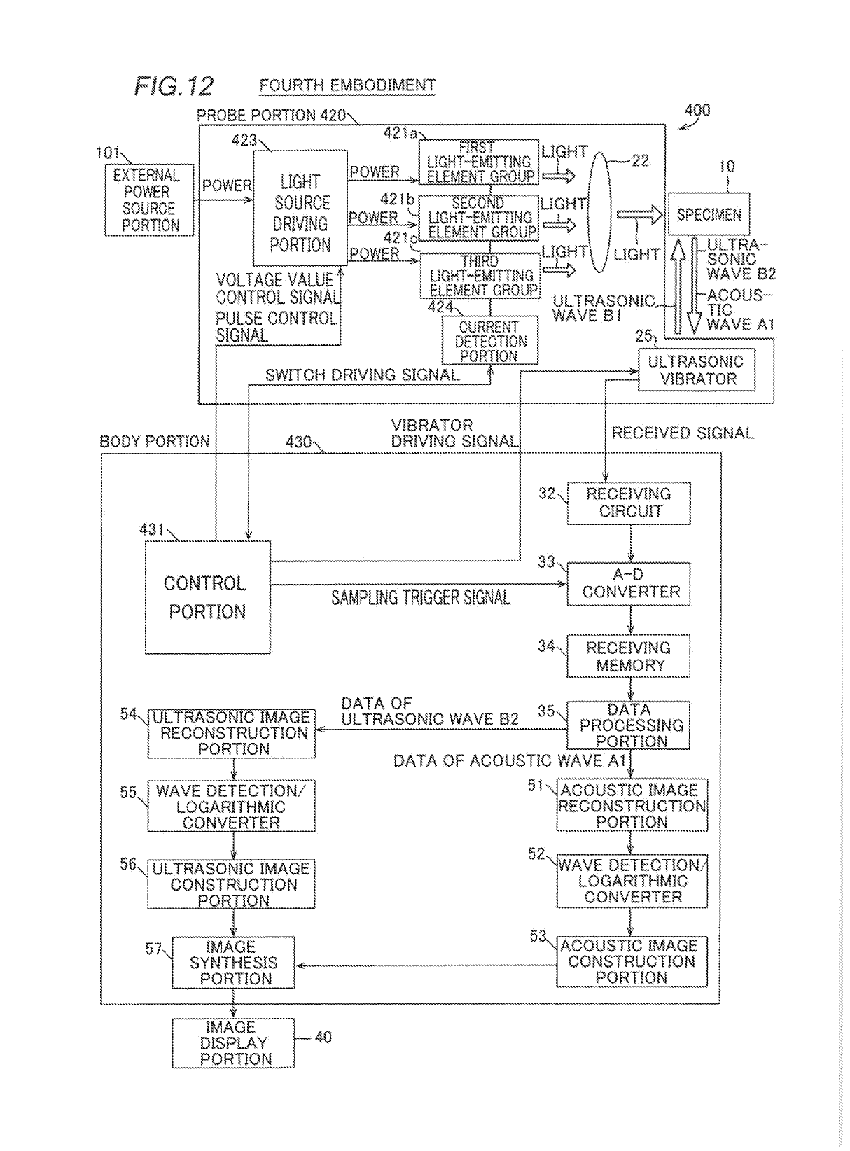

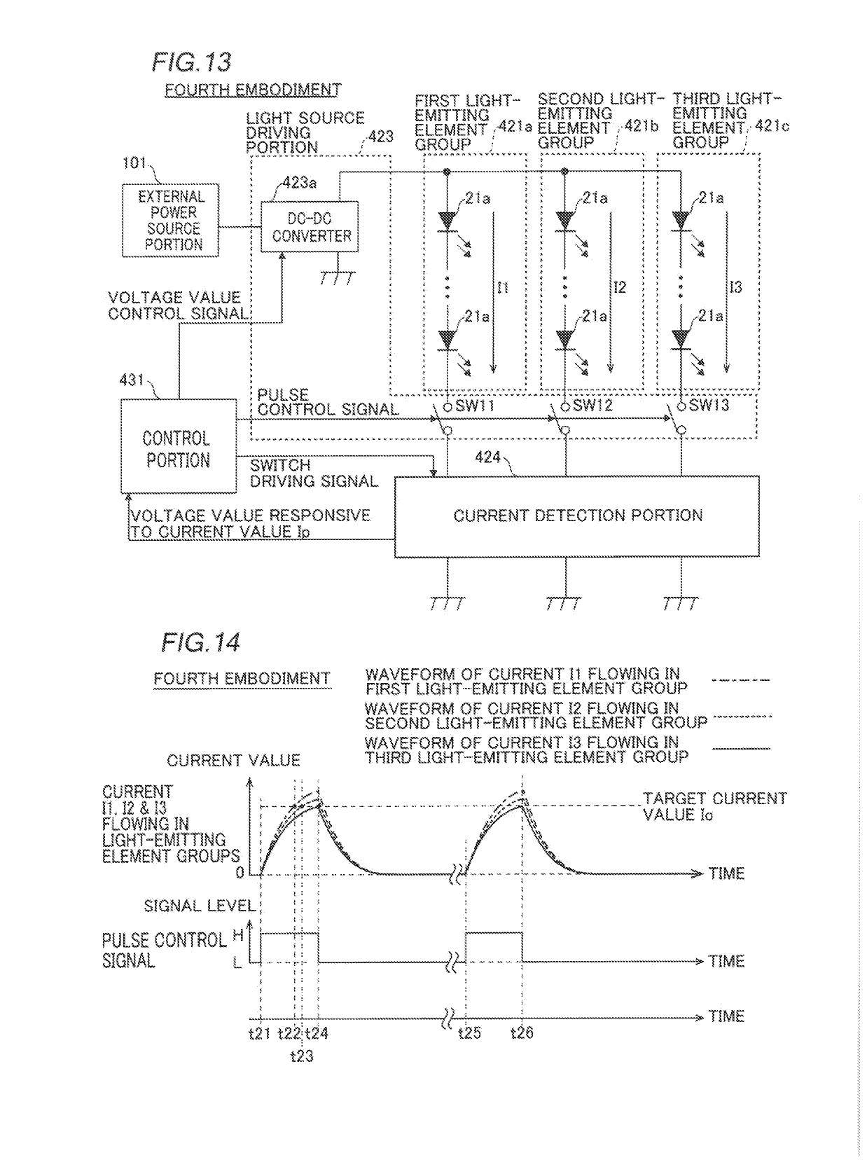

The structure of a photoacoustic imager 400 according to a fourth embodiment of the present invention is now described with reference to FIGS. 12 and 13. According to the fourth embodiment, the photoacoustic imager 400 is provided with a plurality of light-emitting element groups 421a to 421c, dissimilarly to the photoacoustic imagers 100, 200 and 300 according to the first to third embodiments, each provided with one light-emitting element group 21.

As shown in FIG. 12, the photoacoustic imager 400 according to the fourth embodiment is provided with a probe portion 420.

The photoacoustic imager 400 is also provided with a body portion 430. The body portion 430 includes a control portion 431.

According to the fourth embodiment, the probe portion 420 is provided with a first light-emitting element group 421a, a second light-emitting element group 421b, a third light-emitting element group 421c, a light source driving portion 423 and a current detection portion 424, as shown in FIGS. 12 and 13. The first to third light-emitting element groups 421a to 421c are parallelly connected to the light source driving portion 423 respectively.

As shown in FIG. 13, the first to third light-emitting element groups 421a to 421c are configured to include the same numbers of light-emitting diode elements 21a of the same types. In the light-emitting diode elements 21a of the same types, speeds (response speeds) at which current rises upon application of voltage may be dispersed.

The light source driving portion 423 is provided with a DC-DC converter 423a connected to the respective ones of the first to third light-emitting element groups 421a to 421c. The light source driving portion 423 is also provided with a switch portion SW11 configured to be capable of switching conduction and disconnection of the first light-emitting element group 421a and the current detection portion 424. The light source driving portion 423 is also provided with switch portions SW12 and SW13 corresponding to the second and third light-emitting element groups 421b and 421c respectively. The switch portions SW11, SW12 and SW13 are examples of the “driving switch portion” in the present invention.

The current detection portion 424 is configured to be capable of acquiring values of current I1 flowing in the first light-emitting element group 421a, current I2 flowing in the second light-emitting element group 421b and current I3 flowing in the third light-emitting element group 421c. The remaining structures of the photoacoustic imager 400 according to the fourth embodiment are similar to those of the photoacoustic imager 100 according to the first embodiment.

A driving method of the light source driving portion 423 in the photoacoustic imager 400 according to the fourth embodiment is now described with reference to FIG. 14. It is assumed that the response speeds of the first to third light-emitting element groups 421a to 421c are reduced in this order. In other words, it is assumed that the third light-emitting element group 421c has the smallest response speed.

According to the fourth embodiment, the light source driving portion 423 stops supplying power to the first to third light-emitting element groups 421a to 421c at the latest time (t24) among times (t22, t23 and t24) when the values of the current I1, I2 and I3 flowing in the first to third light-emitting element groups 421a to 421c reach a target current value Io, and substantially nulls the current values. This is now more specifically described.

When a pulse control signal is set to a high level at the time t21 (in a state where the values of the current I1, I2 and I3 flowing in the first to third light-emitting element groups 421a to 421c are substantially zero), the values of the current I1, I2 and I3 flowing in the first to third light-emitting element groups 421a to 421c gradually increase respectively.

At the time t22, the value of the current I1 flowing in the first light-emitting element group 421a reaches the target current value Io. In this case, the pulse control signal is kept at the high level. At the time t23, the value of the current I2 flowing in the second light-emitting element group 421b reaches the target current value Io. Also in this case, the pulse control signal is kept at the high level.

At the time t24, the value of the current I3 flowing in the third light-emitting element group 421c reaches the target current value Io. On the basis of that the value of the current I3 flowing in the third light-emitting element group 421c has reached the target current value Io, the pulse control signal is set to a low level. At times t25 to t26, the pulse control signal is set to a high level, similarly to the times t21 to t24.

According to the fourth embodiment, the following effects can be attained:

According to the fourth embodiment, as hereinabove described, the probe portion 420 is provided with the plurality of light-emitting element groups (the first to third light-emitting element groups) 421a to 421c. The plurality of light-emitting element groups 421a to 421c are parallelly connected to the light source driving portion 423 respectively, and the probe portion 420 is provided with the current detection portion 424 acquiring the values of the current I1, I2 and I3 flowing in the plurality of light-emitting element groups 421a to 421c respectively. Further, the light source driving portion 423 is configured to stop supplying power to the plurality of light-emitting element groups 421a to 421c and to substantially null the values of the current I1, I2 and I3 flowing in the plurality of light-emitting element groups 421a to 421c at the time (t24) when the value of the current I3 flowing latest in the third light-emitting element group 421c reaches the target current value Io among the times (t22 to t24) when the values of the current I1, I2 and I3 flowing in the plurality of light-emitting element groups 421a to 421c reach the target current value Io. Thus, the light source driving portion 423 can make the values of the current I1, I2 and I3 flowing in all light-emitting element groups 421a to 421c reach the target current value Io by stopping power supply on the basis of rise of the third light-emitting element group 421c rising latest, also when the speeds (response speeds) at which the current I1, I2 and I3 rises upon application of voltage to the plurality of light-emitting element groups 421a to 421c are dispersed. Further, quantities of light emitted from the plurality of light-emitting element groups 421a to 421c can be ensured, whereby intensity of an acoustic wave A1 can be ensured. Consequently, the photoacoustic imager 400 can more correctly image the acoustic wave A1 by ensuring the intensity thereof.

According to the fourth embodiment, as hereinabove described, the probe portion 420 is provided with the plurality of (first to third) light-emitting element groups 421a to 421c. Further, the switch portions SW11, SW12 and SW13 are provided for the first, second and third light-emitting elements 421a, 421b and 421c respectively. Thus, the photoacoustic imager 400 can properly switch states of applying and not applying light from the first, second and third light-emitting elements 421a, 421b and 421c with the switch portions SW11, SW12 and SW13 provided therefor respectively, also when characteristics (characteristics of forward voltage V_{F}, for example) are dispersed among the plurality of light-emitting elements 421a, 421b and 421c.

The remaining effects of the photoacoustic imager 400 according to the fourth embodiment are similar to those of the photoacoustic imager 100 according to the first embodiment.

Although the present invention has been described and illustrated in detail, it is clearly understood that the same is by way of illustration and example only and is not to be taken by way of limitation, the spirit and scope of the present invention being limited only by the terms of the appended claims.

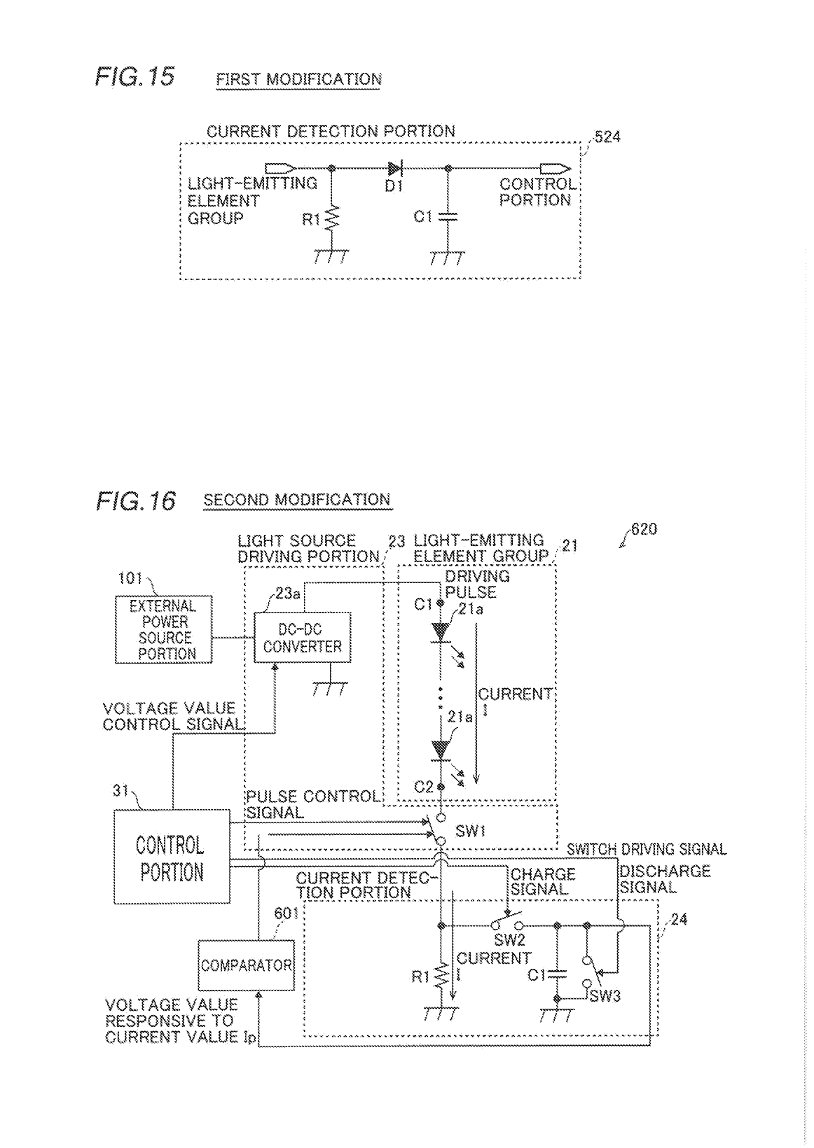

For example, while the current detection portion is configured to maintain the peak of the voltage value responsive to the current flowing in the light-emitting element group by turning on the charge switch portion thereby applying the voltage applied to the detection resistor to the charge capacitor and charging the charge capacitor in each of the aforementioned first and fourth embodiments, the present invention is not restricted to this. According to the present invention, the current detection portion may alternatively be configured to maintain the peak of the voltage value responsive to the current flowing in the light-emitting element group by charging the charge capacitor with a component other than the switch portion. For example, a current detection portion 524 may be configured to maintain a peak of a voltage value responsive to current I flowing in a light-emitting element group by charging a charge capacitor C1 with a diode element D1, as in a first modification shown in FIG. 15.

The current detection portion 524 according to the first modification is provided with a detection resistor R1, the charge capacitor C1 and the diode element D1, as shown in FIG. 15. The anode and the cathode of the diode element D1 are connected to the detection resistor R1 and the charge capacitor C1 respectively. When the current I (having a value Ip) flows in the light-emitting element group, current flows from the side of the detection resistor R1 to the charge capacitor C1, while no current flows from the charge capacitor C1 to the side of the detection resistor R1. Consequently, the current detection portion 524 according to the first modification is capable of maintaining the voltage value responsive to the value Ip of the current I flowing in the light-emitting element group, similarly to the current detection portion 24 according to the first embodiment. A control portion is configured to be capable of acquiring the aforementioned voltage value responsive to the value Ip of the current I flowing in the light-emitting element group.

As hereinabove described, the current detection portion 524 according to the first modification is provided with the detection resistor R1, the charge capacitor C1 and the diode element D1. Thus, the detection resistor R1, the charge capacitor C1 and the diode element D1 can constitute a peak holding circuit. Further, the current detection portion 524 can maintain the peak of the value Ip of the current I flowing in light-emitting diode elements with the charge capacitor C1 and the diode element D1, whereby the same may not control driving of a switch portion dissimilarly to a case of maintaining the peak of the value Ip of the current I flowing in the light-emitting diode elements with the charge capacitor C1 and a switch portion. Consequently, a structure related to control of a photoacoustic imager can be prevented from complication.

While the current detection portion or the voltage detection portion acquires the value of the current flowing in the light-emitting element group and the control portion is configured to determine whether or not the acquired current value has reached the target current value in each of the aforementioned first, second and fourth embodiments, the present invention is not restricted to this. According to the present invention, the photoacoustic imager may alternatively be configured to determine whether or not the value of the current flowing in the light-emitting element group has reached the target current value with a determination means other than the control portion. For example, a photoacoustic imager may alternatively be configured to determine whether or not the value Ip of current I flowing in a light-emitting element group 21 has reached a target current value Io with a comparator 601, as in a second modification shown in FIG. 16.

A probe portion 620 according to the second modification is provided with the comparator 601, as shown in FIG. 16. The comparator 601 is configured to acquire a voltage value corresponding to the value Ip of the current I flowing in the light-emitting element group 21 acquired by a current detection portion 24. A voltage value corresponding to the target current value Io is input in the comparator 601 as reference voltage. Thus, the comparator 601 is configured to be capable of determining whether or not the value Ip of the current I flowing in the light-emitting element group 21 has reached the target current value Io. Further, the comparator 601 is configured to stop supplying power from a light source driving portion 23 to the light-emitting element group 21 by turning off a switch portion SW1 of the light source driving portion 23 when the value Ip of the current I flowing in the light-emitting element group 21 has reached the target current value Io. The switch portion SW1 is an example of the “driving switch portion” in the present invention.

As hereinabove described, the probe portion 620 according to the second modification is provided with the comparator 601 stopping supplying power from the light source driving portion 23 to the light-emitting element group 21 when the value Ip of the current I flowing in the light-emitting element group 21 has reached the target current value Io. Thus, the comparator 601 can substantially null the value Ip of the current I flowing in light-emitting diode elements 21a by automatically stopping supplying power to the light-emitting diode elements 21a on the basis of that the value Ip of the current I flowing in the light-emitting diode elements 21a has reached the target current value Io.

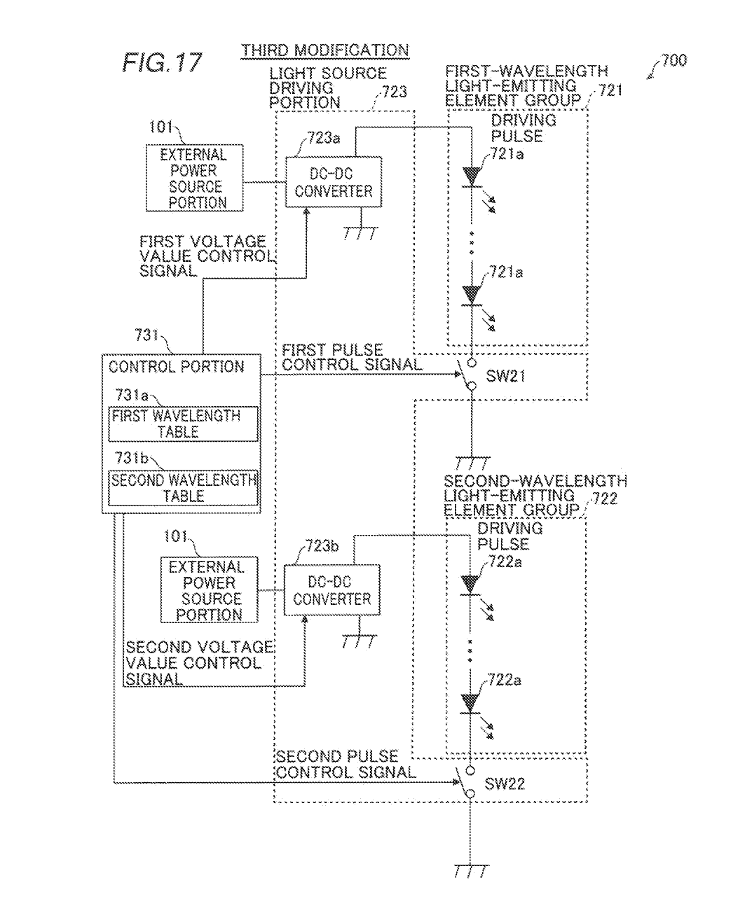

While the single type of light-emitting diode elements emitting light having a wavelength of about 850 nm are employed in each of the aforementioned first to fourth embodiments, the present invention is not restricted to this. According to the present invention, at least two types of light-emitting diode elements may alternatively be employed. For example, a photoacoustic imager 700 may be configured to have two types of light-emitting diode elements, i.e., light-emitting diode elements 721a emitting light having a wavelength of about 850 nm and light-emitting diode elements 722a emitting light having a wavelength of about 760 nm, as in a third modification shown in FIG. 17.

The photoacoustic imager 700 according to the third modification is provided with a first-wavelength light-emitting element group 721, a second-wavelength light-emitting element group 722, a light source driving portion 723 and a control portion 731, as shown in FIG. 17. The first-wavelength light-emitting element group 721 is provided with the plurality of light-emitting diode elements 721a emitting the light having the wavelength of about 850 nm. The second-wavelength light-emitting element group 722 is provided with the plurality of light-emitting diode elements 722a emitting the light having the wavelength of about 760 nm. The light source driving portion 723 is provided with DC-DC converters 723a and 723b and switch portions SW21 and SW22. The control portion 731 stores a first wavelength table 731a and a second wavelength table 731b. The first and second wavelength tables 731a and 731b are configured similarly to the table 331a according to the third embodiment. The light-emitting diode elements 721a emitting the light having the wavelength of about 850 nm are examples of the “first light-emitting element emitting light having a first wavelength” in the present invention. The light-emitting diode elements 722a emitting the light having the wavelength of about 760 nm are examples of the “second light-emitting element emitting light having a second wavelength” in the present invention.

The control portion 731 is configured to transmit a first voltage value control signal and a first pulse control signal corresponding to the first wavelength table 731a as well as a second voltage value control signal and a second pulse control signal corresponding to the second wavelength table 731b to the light source driving portion 723.

As hereinabove described, the photoacoustic imager 700 according to the third modification is provided with the light-emitting diode elements 721a emitting the light having the wavelength of about 850 nm and the light-emitting diode elements 722a emitting the light having the wavelength of about 760 nm, and the light source driving portion 723 is configured to supply a driving pulse to the light-emitting diode elements 721a on the basis of the first wavelength table 731a corresponding thereto and to supply a driving pulse to the light-emitting diode elements 722a on the basis of the second wavelength table 731b corresponding thereto. Thus, the photoacoustic imager 700 may not be provided with current detection portions for acquiring current values Ip respectively also when the same is provided with the light-emitting diode elements 721a and 722a, whereby the light source driving portion 723 can supply driving pulses corresponding to the respective ones of the light-emitting diode elements 721a and 722a while preventing the photoacoustic imager 700 from complication in structure.

While examples of numerical values are shown in order to describe the table and the characteristics of the light-emitting diode elements in the aforementioned third embodiment, the present invention is not restricted to this. According to the present invention, a table may alternatively be constructed with numerical values other than those shown in the aforementioned third embodiment, or light-emitting diode elements having characteristics other than those of the aforementioned ones in the aforementioned third embodiment may alternatively be employed.

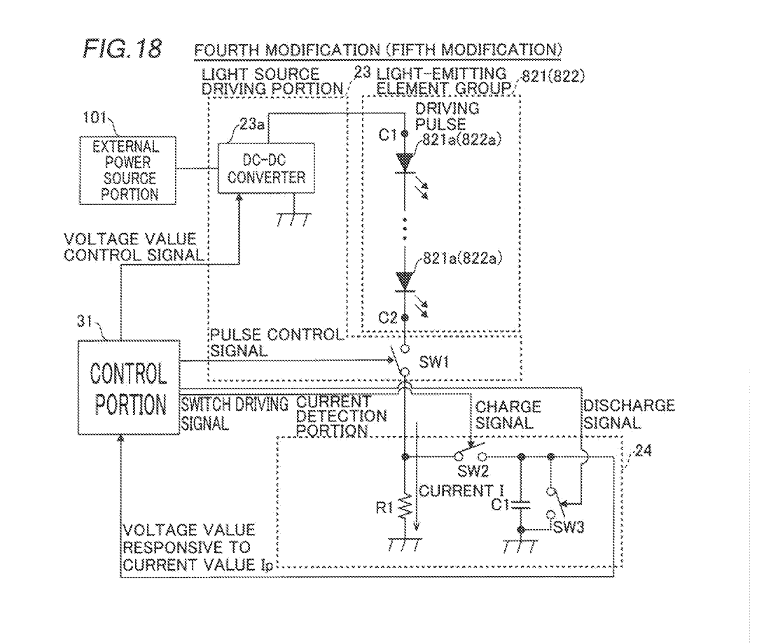

While the light-emitting diode elements are employed as light-emitting elements in each of the aforementioned first to fourth embodiments, the present invention is not restricted to this. According to the present invention, light-emitting elements other than the light-emitting diode elements may alternatively be employed. For example, semiconductor laser elements 821a or organic light-emitting diode elements 822a may be employed as light-emitting elements, as in a fourth or fifth modification shown in FIG. 18.

A light-emitting element group 821 according to the fourth modification is provided with the semiconductor laser elements 821a, as shown in FIG. 18, and configured to be capable of applying light to a specimen. In this case, the semiconductor laser elements 821a can apply laser beams relatively high in directivity as compared with light-emitting diode elements to the specimen, whereby most part of light from the semiconductor laser elements 821a can be applied to the specimen.

On the other hand, a light-emitting element group 822 according to the fifth modification is provided with the organic light-emitting diode elements 822a, as shown in FIG. 18, and configured to be capable of applying light to a specimen from the organic light-emitting diode elements 822a. In this case, the organic light-emitting diode elements 822a can be easily reduced in thickness, and hence the light-emitting element group 822 can be easily miniaturized.

What is claimed is:

1. A photoacoustic imager comprising:
a light-emitting element emitting light to be applied to a specimen;
an acoustic wave detection portion detecting an acoustic wave generated by a detection object in the specimen absorbing the light applied from the light-emitting element to the specimen; and
a light source driving portion supplying power for making the light-emitting element emit the light to the light-emitting element, wherein
the light source driving portion is configured to substantially null the value of current flowing in the light-emitting element by stopping supplying the power to the light-emitting element on the basis of that the value of the current flowing in the light-emitting element has reached a prescribed current value.

2. The photoacoustic imager according to claim 1, wherein
the light source driving portion is configured to start supplying the power to the light-emitting element in a state where the value of the current flowing in the light-emitting element is substantially zero and to stop supplying the power to the light-emitting element on the basis of that the value of the current flowing in the light-emitting element has reached the prescribed current value so that the waveform of the current flowing in the light-emitting element becomes triangular.

3. The photoacoustic imager according to claim 1, wherein
the light source driving portion is configured to change the value of the current flowing in the light-emitting element substantially in all periods for feeding the current to the light-emitting element.

4. The photoacoustic imager according to claim 3, wherein
the periods for feeding the current to the light-emitting element consist of a first period when the value of the current flowing in the light-emitting element increases from a substantially zero state and a second period when the value of the current flowing in the light-emitting element decreases from the prescribed current value.

5. The photoacoustic imager according to claim 4, wherein
the first period corresponds to a rise time of the light-emitting element, and
the second period corresponds to a fall time of the light-emitting element.

6. The photoacoustic imager according to claim 1, wherein
the light-emitting element is configured to emit pulsed light having a triangular waveform by being supplied with the power from the light source driving portion.

7. The photoacoustic imager according to claim 1, further comprising a current detection portion detecting the value of the current flowing in the light-emitting element by detecting the magnitude of a voltage drop in voltage applied to the light-emitting element due to the supply of the power from the light source driving portion.

8. The photoacoustic imager according to claim 7, wherein
the current detection portion is configured to acquire the value of the current flowing in the light-emitting element on the basis of the magnitude of the voltage drop in the voltage and a current characteristic of the light-emitting element corresponding to the magnitude of the voltage drop in the voltage.

9. The photoacoustic imager according to claim 8, wherein
the current characteristic of the light-emitting element corresponding to the magnitude of the voltage drop in the voltage is a characteristic associating a forward voltage value of the light-emitting element and the value of the current flowing in the light-emitting element with each other.

10. The photoacoustic imager according to claim 1, wherein
the light source driving portion is configured to supply a driving pulse based on a table including a voltage value corresponding to the prescribed current value and a pulse width corresponding to the prescribed current value to the light-emitting element.

11. The photoacoustic imager according to claim 10, wherein
the light-emitting element includes a first light-emitting element emitting light having a first wavelength and a second light-emitting element emitting light having a second wavelength different from the first wavelength, and
the light source driving portion is configured to supply the driving pulse to the first light-emitting element on the basis of the table corresponding to the first light-emitting element and to supply the driving pulse to the second light-emitting element on the basis of the table corresponding the second light-emitting element.

12. The photoacoustic imager according to claim 1, wherein
a plurality of the light-emitting elements are provided, and serially connected with each other thereby forming a plurality of light-emitting element groups, and
the light source driving portion includes a plurality of driving switch portions provided on the respective ones of the plurality of light-emitting element groups.

13. The photoacoustic imager according to claim 1, wherein
a plurality of the light-emitting elements are provided, and serially connected with each other thereby forming a plurality of light-emitting element groups,
the plurality of light-emitting element groups are parallelly connected to the light source driving portion respectively,
the photoacoustic imager further comprises a current detection portion acquiring the values of current flowing in the respective ones of the plurality of light-emitting element groups, and
the light source driving portion is configured to substantially null the values of the current flowing in the light-emitting elements by stopping supplying the power to the light-emitting elements at a time when the value of current flowing latest in the light-emitting groups reaches the prescribed current value among times when the values of the current flowing in the respective ones of the plurality of light-emitting element groups reach the prescribed current value.

14. The photoacoustic imager according to claim 1, wherein
the light-emitting element is constituted of a light-emitting diode element.

15. The photoacoustic imager according to claim 1, wherein
the light-emitting element is constituted of a semiconductor laser element.

16. The photoacoustic imager according to claim 1, wherein
the light-emitting element is constituted of an organic light-emitting element.

17. The photoacoustic imager according to claim 1, wherein
the light-emitting element is configured to emit pulsed light having a wavelength in the infrared region.

18. The photoacoustic imager according to claim 1, further comprising a current detection portion detecting the value of current flowing in the light-emitting element, wherein
the current detection portion includes a detection resistor, a capacitor and a detection switch portion.

19. The photoacoustic imager according to claim 1, further comprising a current detection portion detecting the value of current flowing in the light-emitting element, wherein
the current detection portion includes a detection resistor, a capacitor and a diode element.

20. The photoacoustic imager according to claim 1, wherein
the light source driving portion includes a driving switch portion, and
the photoacoustic imager further comprises a comparator configured to stop supplying the power from the light source driving portion to the light-emitting element by turning off the driving switch portion when the value of the current flowing in the light-emitting element reaches the prescribed current value.
